(19) United States
(12) Patent Application Publication
(10) Pub. No.: US 2021/0090735 A1
(43) Pub. Date: Mar. 25, 2021

(54) METHOD FOR EMERGENCY TREATMENT BY ARTIFICIAL INTELLIGENCE

(71) Applicant: National Chiao Tung University, Hsinchu (TW)

(72) Inventors: Ren Shi SHYU, Hsinchu (TW); Lit Min NG, Hsinchu (TW); Shaw Hwa HWANG, Hsinchu (TW); Yu CHIANG, Hsinchu (TW); Bing Chih YAO, Hsinchu (TW); Cheng Yu YEH, Hsinchu (TW); Chih Hung CHIANG, Hsinchu (TW); Kun Ching CHANG, Hsinchu (TW); You Shuo CHEN, Hsinchu (TW); Yao Hsing CHUNG, Hsinchu (TW); Li Te SHEN, Hsinchu (TW); Chi Jung HUANG, Hsinchu (TW); Shun Chieh CHANG, Hsinchu (TW); Ning Yun KU, Hsinchu (TW)

(21) Appl. No.: 16/578,460

(22) Filed: Sep. 23, 2019

(51) Int. Cl. G16H 50/20; G06N 3/08

(52) U.S. Cl. CPC G16H 50/20; G06N 3/08

(57) ABSTRACT

The present invention provides a method fir emergency treatment by artificial intelligence. An artificial neural network is used as the artificial intelligence. Firstly the artificial neural network is trained to make injury classification, inspection list and medical material scheduling correctly. For a patient entering the hospital, the artificial neural network that has been successfully trained is used to accept a plurality of word vectors and various physiological information of the patient to generate an injury classification. The artificial neural network then determines whether the patient has to perform various inspection items respectively with the highest level of the injury classification. The artificial neural network then determines whether the patient needs the various medical materials with the highest level of the injury classification.

### FIELD OF THE INVENTION

The present invention relates to a method for emergency treatment by artificial intelligence, and more particularly to a method for injury classification, inspection list and medical material scheduling by an artificial neural network.

### BACKGROUND OF THE INVENTION

Referring to FIG. 1, which is a schematic diagram for describing the prior art of medical treatment. A patient 1 enters a hospital. A nurse will perform an injury classification 2 at the first stage to obtain a self statement 3 of the patient 1. Then enter the second stage for performing inspection list 4 and medical material scheduling 5 by a doctor. Finally the doctor will perform the necessary medical treatment 6 at the third stage.

Nowadays, AI (Artificial Intelligence) is widely used, and applying the AI method to medical procedures can effectively improve medical efficiency and increase medical accuracy.

### SUMMARY OF THE INVENTION

The object of the present invention is to provide a method for emergency treatment by artificial intelligence, so as to effectively improve medical efficiency and increase medical accuracy. The content of the method for emergency treatment by artificial intelligence according to the present invention is described below.

An artificial neural network is used as the artificial intelligence according to the present invention. Firstly the artificial neural network is trained to learn how to make injury classification, inspection list and medical material scheduling correctly

For a patient entering a hospital, a conversation robot catches a self statement of the patient for converting into a plurality of word strings, and then the plurality of word strings are converted into a plurality of word vectors. Various physiological information of the patient are catched through various wearing devices.

The plurality of word vectors and the various physiological information are inputted into the artificial neural network to generate injury classifications, then take the highest level thereof as the basis for deciding inspection list and medical material scheduling.

The highest level of injury classifications, the plurality of word vectors and the various physiological information are inputted into the artificial neural network, and then various inspection items are inputted into the artificial neural network respectively to produce results that need to be tested or not, and determine whether the patient is to perform the various inspection items respectively.

The highest level of injury classifications, the plurality of word vectors and the various physiological information are inputted into the artificial neural network, and then various medical materials are inputted into the artificial neural network respectively to produce results that require or do not require the medical materials, and determine whether the patient needs the various medical materials respectively.

### BRIEF DESCRIPTION OF THE DRAWINGS

FIG. 1 is a schematic diagram for describing the prior art of medical treatment.

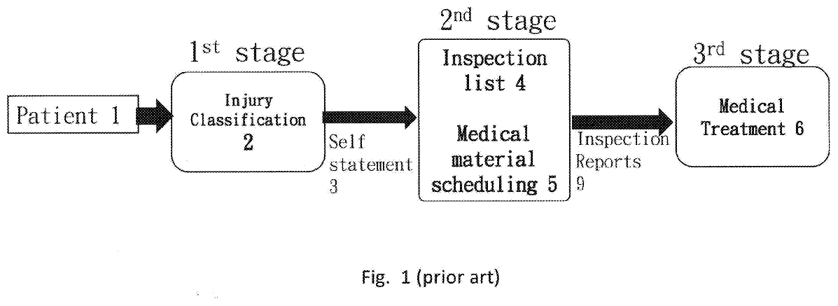

FIG. 2 shows schematically a medical procedure by using an AI according to the present invention.

FIG. 3 shows schematically the model building and the test/prediction of the AI according to the present invention.

FIG. 4 shows schematically the operation of injury classification by an artificial neural network according to the present application.

FIG. 5 shows schematically the operation of inspection list by the artificial neural network according to the present application.

FIG. 6 shows schematically the operation of medical material scheduling by the artificial neural network according to the present application.

### DETAILED DESCRIPTIONS OF THE PREFERRED EMBODIMENTS

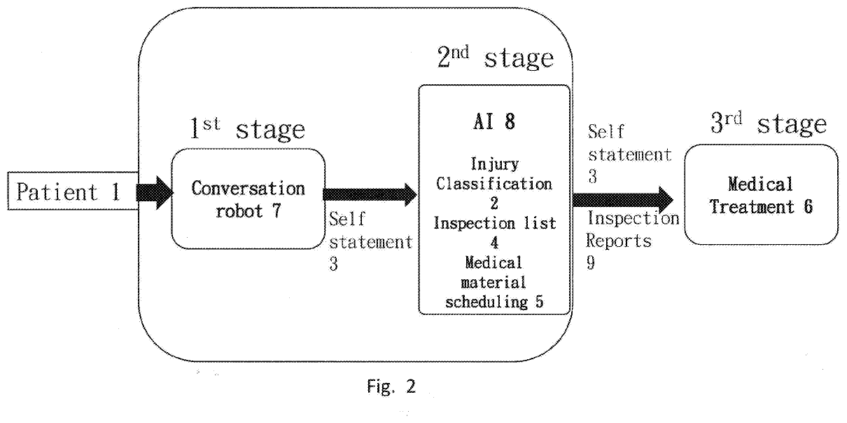

FIG. 2 shows schematically a medical procedure according to the present invention that an AI replaces a nurse to do injury classification 2, and replaces a doctor to do inspection list 4 and medical material scheduling 5.

Referring to FIG. 2, a patient 1 enters a hospital. The first stage and second stage are done by an artificial intelligence (AI). In the first stage, a conversation robot 7 catches a self statement 3 of the patient 1, then enter the second stage, an AI 8 will generate injury classification 2, inspection list 4 and medical material scheduling 5. In the third stage, the self statement 3 of the patient 1 and inspection reports 9 are handed over to the doctor for necessary medical treatment 6.

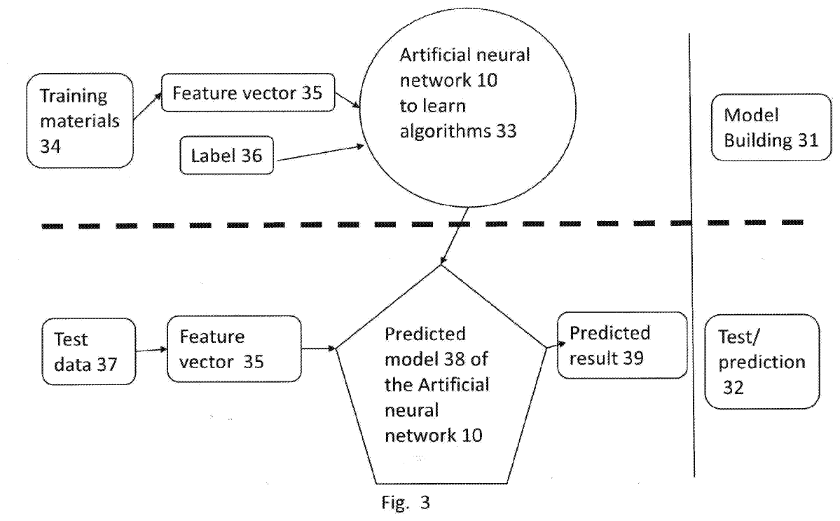

Referring to FIG. 3, a model building 31 and a test/prediction 32 of the AI 8 according to the present invention are described. The AI 8 of the present invention is an artificial neural network 10. The upper part of FIG. 3 shows how to train the artificial neural network 10 to learn an algorithm 33. Correct injury classification 2, inspection list 4 and medical material scheduling 5 are inputted into the artificial neural network 10 respectively as training materials 34, and cooperated with a feature vector 35 and a label 36, so as to let the artificial neural network 10 study how to make injury classification 2, inspection list 4 and medical material scheduling 5 respectively. This is so-called model building 31 stage. The label 36 means injury classification 2, inspection list 4 or medical material scheduling 5.

The lower part of FIG. 3 shows the test/prediction 32 stage. A set of correct injury classification 2, inspection list 4 and medical material scheduling 5 is used as the test data 37 and cooperated with the feature vector 35 for being inputted into a predicted model 38 of the artificial neural network 10, so as to get a predicted result 39. If the predicted result 39 is correct, then the artificial neural network 10 is available for use.

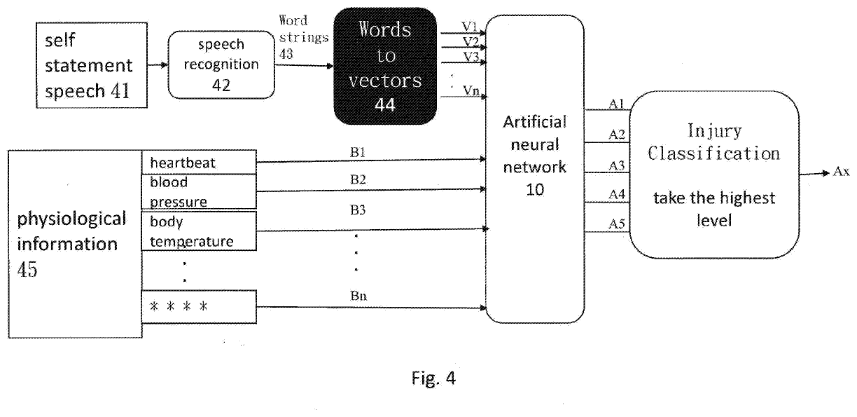

FIG. 4 shows schematically the operation of injury classification by the artificial neural network 10 according to the present application. A patient 1 enters a hospital, then a conversation robot 7 catches a self statement speech 41 of the patient for converting into a plurality of word strings 43 by the speech recognition 42, and then the plurality of word, strings 43 are converted into a plurality of word vectors V1, V2, V3, . . . Vn by the words to vectors 44.

Various physiological information 45 of the patient 1 such as heartbeat value, blood pressure value, body temperature value are catched through various wearing devices to form B1, B2, B3, . . . Bn values.

V1, V2, V3, . . . Vn and B1, B2, B3, . . . Bn are feature vector 35. V1, V2, V3, . . . Vn and B1, B2, B3, . . . Bn are inputted into the artificial neural network 10 to form injury classifications A1, A2, A3, A4, A5, then take the highest level Ax as the basis for deciding inspection list 4 and medical material scheduling 5 stated below.

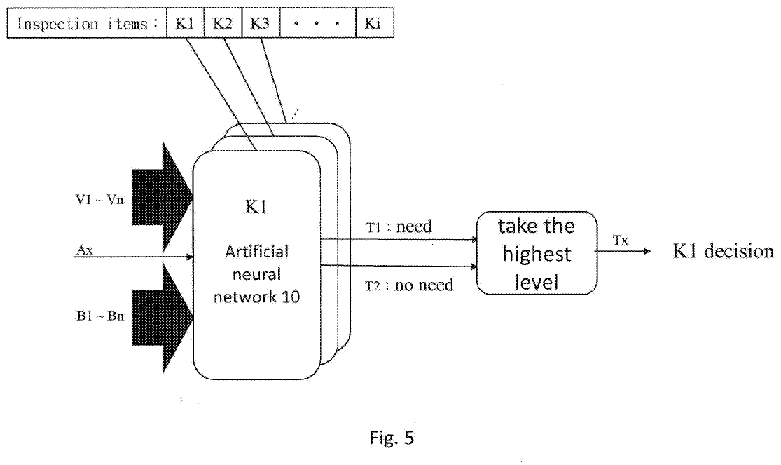

FIG. 5 shows schematically the operation of inspection list by the artificial neural network 10 according to the present application. V1, V2, V3, . . . Vn and B1, B2, B3, . . . Bn and Ax (also a feature vector) are inputted into the artificial neural network 10, and an inspection item K1 is also inputted into to the artificial neural network 10 to generate T1 (need inspection) or T2 (no need inspection), then take the highest level Tx as the basis for deciding if the patient needs to do the inspection item K1.

Similarly the inspection items K2, K3, . . . Ki are inputted into the artificial neural network 10 respectively, and V1, V2, V3, . . . Vn and B1, B2, B3, . . . Bn and Ax are also inputted into the artificial neural network 10 for each, so as to generate T1 (need inspection) or T2 (no need inspection) respectively, then take the highest level Tx as the basis for deciding if the patient needs to do the inspection item K2, K3, . . . Ki respectively.

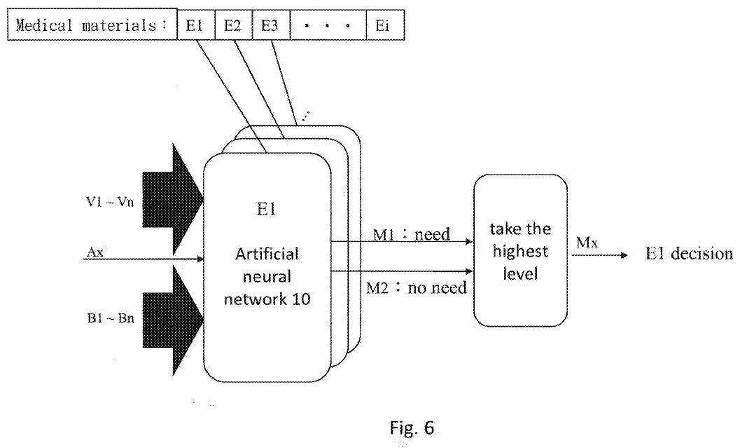

FIG. 6 shows schematically the operation of medical material scheduling by the artificial neural network 10 according to the present application. V1, V2, V3, . . . Vn and B1, B2, B3, . . . Bn and Ax are inputted into the artificial neural network 10, and a medical material E1 is also inputted into to the artificial neural network 10 to generate M1 (need) or M2 (no need), then take the highest level Mx as the basis for deciding if the patient needs the medical material E1.

Similarly the medical materials E2, E3 . . . Ei are inputted into the artificial neural network 10 recpectively, and V1, V2, V3, . . . Vn and B1, B2, B3, . . . Bn and Ax are also inputted into the artificial neural network 10 for each, so as to generate M1 (need) or T2 (no need) respectively, then take the highest level Mx as the basis for deciding if the patient needs the medical materials E2, E3, . . . Ei respectively.

The scope of the present invention depends upon the following claims, and is not limited by the above embodiments.

What is claimed is:

1. A method for emergency treatment by artificial intelligenc, comprising steps as below:
(a) Correct injury classification, inspection list and medical material scheduling are inputted into an artificial neural network respectively as training materials, and cooperated with a plurality of feature vectors and a label, so as to let the artificial neural network study how to make injury classification, inspection list and medical material scheduling respectively;
(b) a conversation robot catches a self statement of a patient for converting into a plurality of word strings, and then the plurality of word strings are converted into a plurality of word vectors;
(c) a plurality of physiological information of the patient are catched through various wearing devices;
(d) The plurality of word vectors and the plurality of physiological information are inputted into the artificial neural network to generate injury classifications, and then take a highest level thereof as the basis for deciding inspection list and medical material scheduling.

2. The method for emergency treatment by artificial intelligenc according to claim 1, wherein the plurality of feature vectors are the plurality of word vectors and the plurality of physiological information, the label is injury classification, inspection list or medical material scheduling.

3. The method for emergency treatment by artificial intelligenc according to claim 1, the highest level of injury classifications, the plurality of word vectors and the various physiological information are inputted into the artificial neural network, and then various inspection items are inputted into the artificial neural network respectively to produce results that need to be tested or not, and determine whether the patient is to perform the various inspection items respectively.

4. The method, for emergency treatment by artificial intelligenc according to claim 1, the highest level of injury classifications, the plurality of word vectors and the various physiological information are inputted into the artificial neural network, and then various medical materials are inputted into the artificial neural network respectively to produce results that require or do not require the medical materials, and determine whether the patient needs the various medical materials respectively.
